(19) United States
(12) Patent Application Publication
(10) Pub. No.: US 2025/0345245 A1
(43) Pub. Date: Nov. 13, 2025

(54) SYSTEM FOR MANAGING DISPENSING OF MEDICATION

(71) Applicants: Roderick L. Brown, Southfield, MI (US); Gregory P. Garland, Novi, MI (US); Dwayne E. Goldsmith, Northville, MI (US); Vassal N. Johnson, Southfield, MI (US); Maurice J. Reeves, Farmington Hills, MI (US)

(72) Inventors: Roderick L. Brown, Southfield, MI (US); Gregory P. Garland, Novi, MI (US); Dwayne E. Goldsmith, Northville, MI (US); Vassal N. Johnson, Southfield, MI (US); Maurice J. Reeves, Farmington Hills, MI (US)

(73) Assignee: ASENDIO, Inc, Ann Arbor, MI (US)

(21) Appl. No.: 19/094,849

(22) Filed: Mar. 29, 2025

Related U.S. Application Data
(60) Provisional application No. 63/644,577, filed on May 9, 2024.

(51) Int. Cl. A61J 7/04; G16H 20/13

(52) U.S. Cl. CPC A61J 7/0454; A61J 7/0418; A61J 7/0436; A61J 7/0445; G16H 20/13; A61J 2200/30; A61J 2200/70; A61J 2205/60; A61J 2205/70

(57) ABSTRACT

The present invention discloses a medication dispensing system. The secure medication dispensing system is configured to dispense Schedule II, III and IV class of drugs that require intricate monitoring for optimized positive utility of the intended users. The system further ensures that pharmaceutics is dispensed in a secure way via multi-factor security through a biometric system including fingerprint, voice, or face recognition. The system further uses a cloud platform to control and regulate dispensing of medication from a medication dispensing device. The system further comprises a controller to open and close medication dispensing compartments. The medication dispensing device uses a specific biometric interface that enables the device to be opened in real time using a defined algorithm. The device dispenses medication in liquid, sublingual film, patch, capsule or tablet form, or any combination, by isolating the medication in unique compartments.

### CROSS REFERENCE TO RELATED APPLICATION

This application claims the priority benefit of the U.S. Provisional Application No. 63/644,577 titled “ASENDIO INTELLIGENT CONTROL SYSTEM-MEDICATION DISPENSING AND ANALYTICSSYSTEM” filed on May 9, 2024. The specification of the above referenced patent application is incorporated herein by reference in its entirety.

### TECHNICAL FIELD OF THE INVENTION

The present disclosure generally relates to a medication dispensing system. More particularly, the invention relates to a secure medication dispensing system for scheduled II, III, and IV classes of drugs that require intricate monitoring for optimized positive utility of the intended users. In addition to ensuring that pharmaceutics are dispensed in a secure way of dispensing of the mentioned drugs via multi-factor security brought forth through a biometric (fingerprint/voice/face recognition), the system also validates location, day/time and operator/physical inspection. The devices fall into the category of Telematics, and has the option of integrating video and other multimedia content for communication. The system includes analytics components that provides business intelligence, predictive analytics and information generated by artificial intelligence applications that provide insight into the use of the system and associated dispensing devices.

### BACKGROUND OF THE INVENTION

Opioids, are a class of drugs used to control pain and can lead to addiction from overuse. Such a unique problem lies in the composition of opioids being from illegal compounds such as heroin and fentanyl, and pharmaceutics such as oxycodone, hydrocodone, codeine, and morphine. It has been conclusively found that when these pharmaceuticals are misused or used illicitly, they can cause respiratory depression, adrenal insufficiency, QT prolongation, low blood pressure and result in addiction. Survey report from United States shows more than nine hundred people a week die from opioid overdoses with rising trend envisaged. Hence, measures/techniques to ensure controlled usage of opioids assumes enormous significance from a practical perspective. Several Schedule III and IV pharmaceutics, such as benzodiazepines, have also been known to cause addiction and contribute to overdose from polysubstance abuse involving opioids. In 2017, over 70,000 individuals died from opioid misuse and over 12,000 died from the misuse of benzodiazepines.

One of the most successful treatment modalities to assist individuals attempting to recover from opioid addiction is Medication Assisted Treatment (MAT). Methadone and Buprenorphine, two of the successful drugs used in MAT are permitted to be dispensed in clinics or other controlled environments by the US Drug Enforcement Administration (DEA) subject to the patients' adherence to attendance and testing requirements to receive take-home doses of this medication. However, patients allowed to take these compounds offsite creates a number of health and safety concerns resulting in strict control and monitoring of this process. Patients that mix pharmaceutics with other substances—such as benzodiazepines—run a higher risk of overdose.

Prevention, which includes informing the public of the danger of drug use and alerting the patients to the likelihood of overdose, are also effective means to mitigate the impact of illicit or misused drugs causing bodily harm.

Controlled applications of poisonous drugs, such as opioids, and management of addictive drugs through appropriate devices/apparatuses etc. have been reflected in a number of select applications as can be envisaged and being used by medication below.

In US4785969A titled, “Medication dispensing system” an improved medication dispensing system is provided for controlled pre-programmed dispensing of medication to a patient and for creating a retrievable patient medication record. The system includes a dispensing unit located, for example, at patient bedside within a hospital room or the like and adapted to receive a pair of medication canisters having magazines with individual cassettes which have been preloaded in a preprogramed manner in the hospital pharmacy or the like respectively to contain scheduled and unscheduled medications for administration to the patient. The dispensing unit is programmed according to individual patient needs to signal a nurse or other personnel at selected times when scheduled medication is prescribed, whereupon the scheduled medication can be accessed for dispensing only after entry of valid nurse or other personnel identification code into a dispensing unit memory. The unscheduled medications may be accessed by the nurse or other personnel in a similar manner but on an untimed basis as required by the patient. The dispensing unit further includes sensors for detecting removal of any medication cassette and for signalling the dispensing unit memory to create a corresponding patient medication record. A data transmission device is provided for selectively programming the dispensing unit memory and/or for reading the patient medication record from the memory.

In the French application, WO2008086628A1 titled, “Medication dispensing system and method” a medication dispensing system and method where the patient can have his personal console capable of receiving an insert having a tray with an array of medication compartments corresponding to respective times and dates at which the medication is prescribed to be taken is discussed. A database in the console can be set with the specified times and dates at which the medication is to be taken. When the console determines the time to dispense medication in one of the compartments has been reached, a visual and/or audible indicator can be triggered. Membranes which are to be hand-removed by the patient cover the respective compartments. A detector provided under the insert can detect across the tray whether or not the membrane corresponding to the selected medication compartment has been removed.

In US5879699A titled, “Medication dispensing system” a hollow, consumable fillable dispenser having a filler tube for delivery of medication in a more palatable manner is discussed. The dispenser may be a hard or chewy confection and defines a hollow chamber which may be filled with a medication at time of use by means of the filler tube.

In US5047948A titled, “Medication dispensing system” a medication dispensing system is discussed that comprises of a portable, securable medication cabinet which includes a programmable logic controller to provide for patient medication control. The system includes peripheral components such as a transmitter, patient response means, display means, and a clock. The securable medication cabinet permits easy access by the patient to medication which is prescribed and dispensed logically. The securable cabinet restricts access by the patient to unauthorized medication. The securable medication cabinet contains a plurality of medication dispensing components, each comprising dispensing means, such as a coil attached to a motor positioned interiorly of a wall of the cabinet, to hold a medication package and dispense the package upon actuation of the dispensing means. A receptacle in the bottom of the cabinet receives a medication package as it is dispensed. A door in a side wall of the cabinet permits access by the patient to a medication package in the receptacle, but a stop in the cabinet restricts opening of the door so that the patient may not reach unauthorized medication packages. The programmable logic controller accepts prescription data and accepts and evaluates patient physical data. The controller is interfaced with; (1) the medication dispensing components to select a dispensing component in re prescription data and patient physical data, (2) display components to indicate response by patient to a signal entered; (3) interfaced with a peripheral system to respond to a reaction of patient.

In yet another invention, US20090057183A1 titled “Medication Dispensing System” discusses a medication dispensing system which has only two sheet components; a substantially transparent front sheet that has containers for medications formed into it. The intersection of the container with the front sheet defines an opening of each container, and the opening has a line of weakness allowing the container to be broken loose from the remaining plane portion of the front sheet. The front sheet is permanently affixed to a stiff, non-frangible backing sheet, after the containers are loaded with medications. The backing sheet has indicia on one or both sides for forms to be filled in by a dispensing professional concerning the various medications held in the containers, and also indicia in registry with the containers to indicate a day of the week and a time to a patient, so that the patient can remove the correct container and take the medications contained in it.

Further, few conventional components and devices (100, 200, 250) used for medication management are disclosed in FIG. 1 to FIG. 3.

While all these devices/apparatuses focus on smooth dispensing of medicines, none of these have security features embodied to prevent overuses, especially when dispensing of medicines needed to be on a secured basis as of Schedule II & III drugs. Neither do these systems work in an integrated fashion to predict trends or do they estimate likelihood of overdose given the demographic composition of the patient.

To ensure the security of the medication dispensing, the present invention employs exclusive and unique safety features while retaining the smoothness and elasticity of dispensing thus scoring well over similar inventions. The system is programmed, controlled and interfaces to a control device located at the point of distribution. This control device is connected via the internet network to a central cloud controller as well.

### SUMMARY OF THE INVENTION

The present invention discloses a medication dispensing system. The invention is comprised of three tiers. Including a Tier 1 cloud software system that serves to manage devices located in remote clinics and offices, a Tier 2 control system that is a server located in these remote offices, and a Tier 3 Internet of Things (IoT) device, which dispenses medication and communicates with the patient/client. The Tier 1 Cloud Software is called the Controlled Substance Management (CSM) PolyCloud Recovery Platform (CSMPRP).

Tier 1 is a cloud software system, comprised of nine elements including IoT Controller Management Software, cloud infrastructure, one or more applications, private social network, analytics algorithms, AI applications, database, dashboards and clinic management processes and policies. The IoT Controller Management Software controls, manages, and backs up the devices on the client premise. This device also stores back up files of fingerprints and other biometric data. The cloud infrastructure includes Public (Microsoft/Amazon) elements and Private server elements. The applications enable interaction with clients and clinicians based on data captured from the system. The private social network facilitates therapeutic communication between clients and clinicians. The analytic algorithms support the recovery process. The Multimedia TV application manages and stores video content for broadcast over the network and directly to the patients and clients. The database of drug sample test results could be aggregated in a de-identified manner. The dashboards are provided for access and reporting.

Tier 2 controller is a server that communicates directly with Tier 1 software and Tier 3 IoT devices. Tier 2 stores biometric data, enables the programming of the medication dispensing devices, and generates messages for clinicians. This system is a server that stores rules that govern analysis and programming, stores content and maintains the programs to control devices. This is a Wi-Fi enabled device that must be used to interface with Tier 3 device. This Tier also includes processing capability that can be locally deployed for analytics and AI functions.

Tier 3 device provides safe, secure, and controlled dispensing of pharmaceuticals via a Multi-dimensional Secure Medication Dispensing Procedure. Specifically, the device limits the medication delivery to one dose at prescribed times. The device limits access to only the prescribed patient, and that medication is accessible at a previously specified location, typically at the patient's place of residence. This invention also records distribution at the time of delivery using video technology and records instances where unauthorized individuals attempt to access the device. The device provides feedback that indicates the psychosocial state of mind of the client. Selected models of the device provide access to multiple doses of medication and tracks appropriate usage. The device provides audible indications if an inappropriate dose of medication is selected. Tier 3 devices also have the capability to store multiple doses of medication that can be remotely dispensed using Tier 1 software applications. This enables a three-factor, controlled dispensing of medication at a specific geographic location, by a biometrically identified licensed professional, in concert with a licensed individual located in a remote central location.

This secure device provides a single dose of medication in liquid, sublingual film, patch, capsule, or tablet form—or any combination—by isolating the medication in unique compartments. The device will provide an audible alert when it's time for the medication. The device will also provide a second audible alert when someone other than the authorized user is trying to access the medication. A third alert feature is sounded if the cover to the medication system is opened and left open for a period of time greater than two minutes.

The device recognizes the authorized patient by employing biometric identification, which is only capable of opening the device at specific remote locations. Cellular capability is employed to broaden the remote location. As an audit trail, the device is designed to recognize the container that holds the dose. Subsequent doses of medication are only delivered after the previous container is deposited in the device. Legislative rules require that containers that deliver medication are returned to the prescribing facility. These containers are marked with labels that specify the drug compound and the individual specified to receive the dose of medication. The device tracks successful operations of the delivery and attempted access. The device tracks and notifies operators (via audible alert system) if an unauthorized individual attempts to access medication in the device, or if the device is removed from the previously authorized dispensing location. The device can be geographically located using Global Positioning System (GPS), Wi-Fi, utility meter integration or other telecommunications protocols should the location become unknown to the patient or prescribing facility. The device can optionally track the containers used to dispense medication.

The present invention discloses a system for managing dispensing of medication. The system comprises one or more dispensing devices and one or more Internet of Things (IoT) devices in communication with the dispensing devices. The dispensing device is configured to store and dispense different types and doses of medication. The dispensing device is configured to receive biometric data, identification data of a user including a patient and a caregiver, image data, video data, location data of dispensing device and dosage data of patient.

The IoT device is configured to store biometric data and identification data of a caregiver and a patient, and location data. The IoT device is configured to enable the dispensing device to provide a dose of medication to the patient at a predefined time on receiving authentication of biometric data, identification data of at least one of patient and caregiver data, and location data of dispensing device.

The dispensing device comprises a housing comprising one or more compartments and a door to provide access within the housing. Each compartment comprises at least one lid. The dispensing device further comprises a latching device configured to lock and unlock the door, and a metal strip comprises an electronic connection point to detect tampering of the dispensing device. In the open position of the door, the metal strip holds the door in place. When the latching device is actuated to close the door, the metal strip releases the door, allowing the door to close the housing using a solenoid connected to the latching device. The dispensing device is configured to dispense medication in the form of liquids, tablets, pills, sublingual films, or capsules.

The dispensing device further comprises one or more sensors in communication with a control device. The sensors comprises a biometric sensor configured to capture the biometric data by at least one of voice recognition, fingerprint scanning, retina scanning and facial recgnition. In one embodiment, the biometric sensor comprises a touch pad including a finger print scanner configured to capture fingerprint data.

The dispensing device further comprises at least one camera device disposed at the housing of the dispensing device. The camera device is in communication with the control device. The camera device is configured to capture image data including images of compartments to record available doses of medication at the compartments upon opening and closing the door.

The dispensing device further comprises at least one video device in communication with the control device. In one embodiment, the video device is disposed at the housing of the dispensing device. The video device is configured to capture video data including videos of environment of the dispensing device on unauthorized access of the dispensing device. The dispenser device further comprises at least one GPS module in communication with a control device. The GPS module is configured to capture location data.

The dispensing device further comprises a switch in communication with the control device and the latching device. The dispensing device further comprises at least one memory in communication with the control device and a powersource to operate the dispensing device. The memory is configured to store the biometric data, image data, video data, and location data of the user.

The control device is configured to receive and store biometric data, image data and video data of the user, and location data of the dispensing device. The control device is further configured to transmit the biometric data, image data and video data of the user, and location data of the dispensing device to the server for registration of the user and for authentication on accessing the dispensing device. The dispensing device is further configured to provide verbal reminders and step-by-step instructions to assist patients with medication adherence.

The system further comprises at least one database configured to store patient data including, but not limited to, biometric data, identification data, medical data and dosage data, location data comprising information related to location approved for operation of the dispensing device, and caregiver data including biometric data and identification data. In one embodiment, the biometric data and identification data includes, but not limited to, voice data, image data and video data. The image data includes images of compartments to record available doses of medication at the compartments upon opening and closing the dispensing device. The video data includes videos of environment of the dispensing on unauthorized access of the dispensing device.

The system further comprises a server in communication with the dispensing devices, IoT devices and the database. The server is configured to receive patient data and caregiver data from the dispensing devices and link the patient data and caregiver data to respective unique identities. The server is further configured to receive dosage data of patients. The server is further configured to receive image data and video data of the user on accessing the dispensing device.

The server is further configured to compare the identification data and biometric data of the user with stored identification data and biometric data for sending authentication to IoT devices and dispensing devices for dispensing medication at predefined time. The server is configured to analyze image data and dosage data, and determine adherence of patient to dosage compliance. The dosage compliance is determined by determining if the compartments are same as that of an initial number of compartments, and by determining the amount of medication before accessing the device and after the closure of the dispensing device.

The server is configured to manage video data for broadcasting to respective user including patient, healthcare provider or caretaker. The server is configured to provide a dashboard comprising patient data and dosage data to enable patients and caretakers to track the information of medication. In one embodiment, the dispensing device comprises a multi-factor authentication system.

The server is configured to track whether the patient takes medication as prescribed by analyzing patient data, dosage data, and image data from the dispensing device. The server is further configured to generate and transmit an alert to healthcare providers on non-adherence of dosage compliance. The server is further configured to automatically record dispensing events and transmit dosage compliance data to an external system for healthcare provider review.

The server is further configured to enable remote monitoring of patient adherence by healthcare providers by providing access to medication adherence data. The server is further configured to allow healthcare providers to intervene on detection of non-adherence of dosage compliance by generating alerts to the patient and caregiver.

The server is further configured to detect risk behaviors, including skipped doses and patterns indicating misuse, by analyzing patient medication adherence data. The server is further configured to generate and transmit alerts to healthcare providers on detecting risk behaviors, enabling timely intervention and monitoring. The server is further configured to verify if location data of the dispensing device matches against predefined location data before authorizing the release of medication. The server is further configured to ensure that the medication dispensing activities align with prescription control measures mandated by regulatory authorities.

The server is configured to use artificial intelligence to provide tailored guidance on adherence strategies and motivation. The server enables patients to immediately connect with healthcare providers in case of distress via a user device in communication with the server. The server is further configured to lock the dispensing device on detection on attempts of unauthorized access of the dispensing device. The server enables patients and healthcare providers to customize alerts via text, email, or phone calls. The server is configured to verify if the temperature and environmental conditions of the dispensing devices match predefined safe storage parameters before authorizing medication storage. The server is configured to transmit an alert to the users if the stored medication exceeds the safe storage parameters.

The dispensing device further comprises a user interface in communication with the control device. The user interface is configured to display clear instructions, alerts, and patient information in a user-friendly format. In one embodiment, the dispensing device is configured to support multiple languages for better accessibility. The system further comprises one or more user devices associated with a respective user. The user devices are in communication with the server. The server is configured to enable patients, healthcare providers and authorized caregivers to monitor adherence and set up reminders via the user device.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a Microcomputer, Raspberry Pi, or similar device, which is the controller that is used for the program.

FIG. 2 is a typical security box used as input to the production process.

FIG. 3 is a component used to capture digital identification of a user.

FIG. 4 illustrates a medication dispensing device having a single compartment for dispensing various types of medication, according to an embodiment of the present disclosure.

FIG. 5 illustrates a medication dispensing device having at least three compartments for dispensing different types of medication, according to an embodiment of the present invention.

FIG. 6 illustrates a medication dispensing device having at least six compartments for dispensing different types of medication, according to another embodiment of the present invention.

FIG. 7 shows the modular system components that comprise the physical and electronic elements within the dispensing devices, according to an embodiment of the present invention.

FIG. 8A depicts a perspective view of a dispensing device in an open position showing a two by four array of storage compartment, according to an embodiment of the present invention.

FIG. 8B depicts an enlarged 3D perspective view of a single compartment of the dispensing device, according to an embodiment of the present invention.

FIG. 8C depicts a front view of the compartment of FIG. 8A.

FIG. 9 depicts an electronic design component and the bus used to connect these components, according to an embodiment of the present disclosure.

FIG. 10 provides a block view of the input screen that is used to program the six-dimensional secure medication dispensing device, according to an embodiment of the present invention.

FIG. 11 is a logic flowchart representing the process of verification of the day of the week once the location, fingerprint and/or facial verification and/or voice recognition is completed, for opening the particular dispensing compartment, according to an embodiment of the present invention.

FIG. 12A illustrates a perspective view of a two-dose modified Polycase container assembly, according to an embodiment of the present invention.

FIG. 12B illustrates an exploded view of the two-dose modified Polycase container assembly having two doors and dose holders, according to an embodiment of the present invention.

FIG. 12C illustrates a perspective view of a dose bottle holder of the two-dose modified Polycase container assembly, according to an embodiment of the present invention.

FIG. 13A illustrates a perspective view of a standard metal box having a seven to fourteen-dosage unit, according to an embodiment of the present invention.

FIG. 13B illustrates a perspective view of the fourteen-dosage unit with a pull up chamber discharge design of the standard metal box, according to an embodiment of the present invention.

FIG. 13C illustrates a perspective view of a 3D printed internal working of seven dose units with a pull up chamber discharge design of the standard metal box, according to an embodiment of the present invention.

FIG. 13D illustrates a perspective view of fourteen dose units with a pull up chamber of the standard metal box and a servo motor, according to an embodiment of the present invention.

FIG. 13E illustrates a perspective view of a seven-dose pusher device using a pull up chamber discharge design and 3D printed base, according to another embodiment of the present invention.

FIG. 14A illustrates a perspective view of a remote monitoring device, according to an embodiment of the present invention.

FIG. 14B illustrates a perspective view of an interior tray of the remote monitoring device, according to an embodiment of the present invention.

FIG. 14C illustrates a perspective view of a channel having a vending machine coil and motor, according to an embodiment of the present invention.

FIG. 14D illustrates a perspective view of a tray comprising a seven-dose unit, according to an embodiment of the present invention.

FIG. 14E illustrates a rear view of a tray comprising a bottom discharge portion, according to an embodiment of the present invention.

FIG. 14F illustrates a perspective view of a tray comprising a fourteen dose unit, according to an embodiment of the present invention.

FIG. 14G illustrates an alternative design case for transportation having five pipes with vending machine coils, according to another embodiment of the present invention.

FIG. 14H illustrates a perspective view of the vending machine motor and coil enclosed inside a PVC pipe and elbow, according to another embodiment of the present invention.

FIG. 15 illustrates a perspective view of a twenty-eight-dose device housed in a metal enclosure, according to an embodiment of the present invention.

FIG. 16 illustrates a perspective view of a standard metal cash box with a video screen and microphone, according to an embodiment of the present invention.

FIG. 17A illustrates a perspective view of a single carousel design placed in a polycase container, according to an embodiment of the present invention.

FIG. 17B illustrates a perspective view of a dual carousel design placed in a polycase container, according to an embodiment of the present invention.

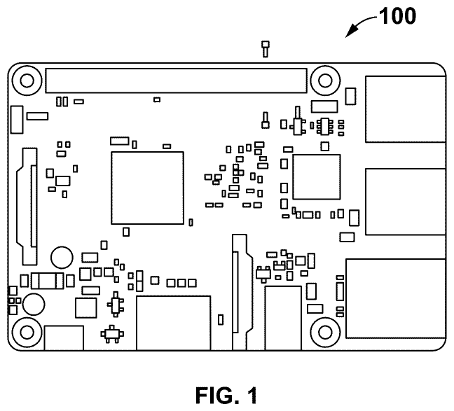

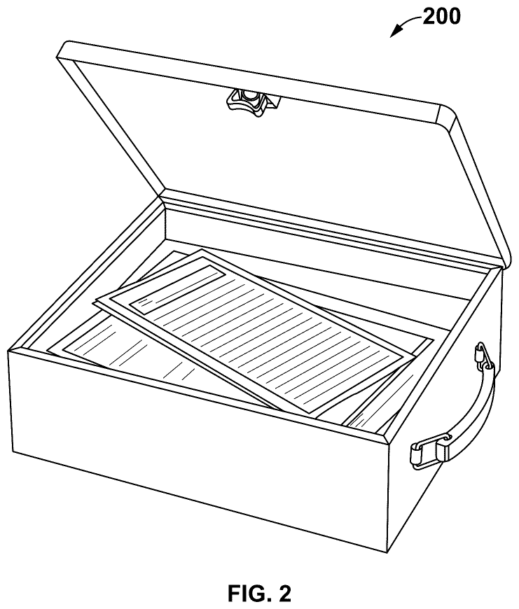

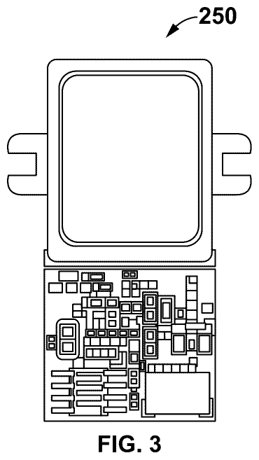

### DETAILED DESCRIPTION OF THE PREFFERED EMBODIMENTS

The following disclosure describes the dispensing devices, systems and methods which constitute the present invention. The elements of the invention are defined by referring to standard electronic and physical components. However, the elements used to define the invention combined with the skill in the art, is readily applied to other systems and methods. The invention is comprised of software in a cloud host system (CSMPRP), server software that interfaces with the IoT devices, and software embedded along with mechanical components that comprise the physical IoT secure medication dispensing system. The system also includes a cloud-based interface that syncs the device via appropriate programming of software to identify specific operating parameters, retrieve usage information, and to pinpoint the location of the device. A detailed disclosure of the devices, systems and methods of the present invention for administration of a drug dosage are provided herein below.

The term “systems that include a drug dosage form and a dispensing device' as used herein refers to a three-tier drug dispensing system for delivery and monitoring of drug administration. A system of the invention may be used to monitor and deliver both efficacious and maximum dosages such that the amount of drug delivered, corresponding efficacy, and safety are enhanced over currently available systems. The system may also have one or more features that provide for improved safety and ease of use over currently available systems including security features that prevent unauthorized access to the stored drugs, a dispensing dose lock-out feature, a dose counting feature, a memory means for retaining information about dose delivery, identity of the authorized user and an interface for bidirectional exchange of information with a user or another device such as a computer.

The system ensures controlled access to take-home medication, dispensing doses only at prescribed times to prevent early use or diversion. For example, the system is configured to release a single methadone dose for Sunday only when it is due, preventing Saturday overdoses. The system is configured to protect medication against unauthorized access, theft, or tampering. The biometrically locked compartments are enabled to allow authorized access at the scheduled time. The system ensures that only the designated patient accesses the medication using biometrics or multi-factor authentication. The system tracks whether patients take their medication as prescribed and alerts healthcare providers if doses are skipped or misused. The system sends a real-time notification to the clinic if a patient fails to retrieve their Sunday dose. The system automatically records dispensing events and transmits compliance data to Medication Assisted Treatment (MAT) clinic systems like Methasoft for clinician review. The system includes Cloud-based data synchronization that logs when a dose is dispensed or if there is an unauthorized attempt to access the device.

The system enables clinicians and MAT providers to remotely monitor patient adherence and intervene when necessary. The system is configured to provide mobile app dashboard for healthcare providers that flags non-compliance cases. The system detects and alerts clinicians about potential risk behaviors like skipped doses or patterns that suggest diversion or misuse. For example, if a patient tries to access medication before the scheduled time, an alert is sent to clinic staff. The system ensures medication access is limited to approved locations, such as the patient's home, to prevent misuse in high-risk areas. For example, GPS tracking ensures that the device only functions within a defined geographical zone. The system uses data trends to predict high-risk behaviors, relapses, or non-adherence, allowing proactive intervention. The system flags patients at risk of missing doses based on past adherence patterns.

The system is configured to provide verbal reminders and step-by- step instructions to assist patients with medication adherence. For example, the system includes a voice assistant that issues prompts such as, “It's time for your medication. Please scan your fingerprint.” The system includes an LED display or touchscreen interface to present instructions, alerts, and patient information in a user-friendly format. For example, a display may show “Next Dose Available in 4 Hours” with icons for easy navigation. The system is configured for mobile application integration, enabling patients or authorized caregivers to monitor adherence and configure reminders. For example, the system allows patients to check their medication schedule via an application or user device.

The system supports multi-language functionality, allowing users to select a preferred language for improved accessibility. For example, the system is configured to provide on-screen and audio instructions in English, Spanish, or French. The system incorporates AI-based adherence coaching, wherein an artificial intelligence module provides tailored guidance on adherence strategies and motivation. For example, the system may issue recommendations such as, “Try setting an alarm 5 minutes before your dose time to improve consistency.”

The system is configured to integrate emergency contact functionality, allowing patients to connect with a counselor or clinician in distress situations. For example, if a patient misses two consecutive doses, the system prompts the patient to contact their MAT provider. The system includes a battery backup mechanism to ensure uninterrupted functionality during power outages. For example, a built-in lithium battery provides 24-hour backup power to maintain operational status. The system incorporates a tamper detection and self-locking mechanism to detect unauthorized access attempts. For example, if an unauthorized individual attempts to force open or disable the system, it initiates a lockdown and transmits an alert to the clinic. The system enables customized alerts and notifications, allowing patients and providers to configure notifications via text message, email, or phone calls. For example, a patient may receive a text alert if they fail to access their medication within 10 minutes of the scheduled time. The system is configured with temperature and environmental monitoring functionality to ensure that medications are stored within a safe temperature range. For example, the system is programmed to transmit an alert to the clinic if the stored medication is exposed to excessive heat.

The system comprises one or more dispensing devices and one or more Internet of Things (IoT) devices in communication with the dispensing devices. The dispensing device is configured to store and dispense different types and doses of medication. The dispensing device is configured to receive biometric data, identification data of a user including a patient and a caregiver, image data, video data, location data of dispensing device and dosage data of patient.

The IoT device is configured to store biometric data and identification data of a caregiver and a patient, and location data. The IoT device is configured to enable the dispensing device to provide a dose of medication to the patient at a predefined time on receiving authentication of biometric data, identification data of at least one of patient and caregiver data, and location data of dispensing device.

The dispensing device comprises a housing comprising one or more compartments and a door to provide access within the housing. Each compartment comprises at least one lid. The dispensing device further comprises a latching device configured to lock and unlock the door, and a metal strip comprises an electronic connection point to detect tampering of the dispensing device. In the open position of the door, the metal strip holds the door in place. When the latching device is actuated to close the door, the metal strip releases the door, allowing the door to close the housing using a solenoid connected to the latching device. The dispensing device is configured to dispense medication in the form of liquids, tablets, pills, sublingual films, or capsules.

The dispensing device further comprises one or more sensors in communication with a control device. The sensors comprises a biometric sensor configured to capture the biometric data by at least one of voice recognition, fingerprint scanning, retina scanning and facial recgnition. In one embodiment, the biometric sensor comprises a touch pad including a finger print scanner configured to capture fingerprint data.

The dispensing device further comprises at least one camera device disposed at the housing of the dispensing device. The camera device is in communication with the control device. The camera device is configured to capture image data including images of compartments to record available doses of medication at the compartments upon opening and closing the door.

The dispensing device further comprises at least one video device in communication with the control device. In one embodiment, the video device is disposed at the housing of the dispensing device. The video device is configured to capture video data including videos of environment of the dispensing device on unauthorized access of the dispensing device. The dispenser device further comprises at least one GPS module in communication with the control device. The GPS module is configured to capture location data.

The dispensing device further comprises a switch in communication with the control device and the latching device. The dispensing device further comprises at least one memory in communication with the control device and a powersource to operate the dispensing device. The memory is configured to store the biometric data, image data, video data, and location data of the user.

The control device is configured to receive and store biometric data, image data and video data of the user, and location data of the dispensing device. The control device is further configured to transmit the biometric data, image data and video data of the user, and location data of the dispensing device to the server for registration of the user and for authentication on accessing the dispensing device. The dispensing device is further configured to provide verbal reminders and step-by-step instructions to assist patients with medication adherence.

The system further comprises a database configured to store patient data including, but not limited to, biometric data, identification data, medical data and dosage data, location data comprising information related to location approved for operation of the dispensing device, and caregiver data including biometric data and identification data. In one embodiment, the biometric data and identification data includes, but not limited to, voice data, image data and video data. The image data includes images of compartments to record available doses of medication at the compartments upon opening and closing the dispensing device. The video data includes videos of environment of the dispensing on unauthorized access of the dispensing device.

The system further comprises a server in communication with the dispensing devices, IoT devices and the database. The server is configured to receive patient data and caregiver data from the dispensing devices and link the patient data and caregiver data to respective unique identities. The server is further configured to receive dosage data of patients. The server is further configured to receive image data and video data of the user on accessing the dispensing device.

The server is further configured to compare the identification data and biometric data of the user with stored identification data and biometric data for sending authentication to IoT devices and dispensing devices for dispensing medication at predefined time. The server is configured to analyze image data and dosage data, and determine adherence of patient to dosage compliance. The dosage compliance is determined by determining if the compartments are same as that of an initial number of compartments, and by determining the amount of medication before accessing the device and after the closure of the dispensing device.

The server is configured to manage video data for broadcasting to respective user including patient, healthcare provider or caretaker. The server is configured to provide a dashboard comprising patient data and dosage data to enable patients and caretakers to track the information of medication. In one embodiment, the dispensing device comprises a multi-factor authentication system.

The server is configured to track whether the patient takes medication as prescribed by analyzing patient data, dosage data, and image data from the dispensing device. The server is further configured to generate and transmit an alert to healthcare providers on non-adherence of dosage compliance. The server is further configured to automatically record dispensing events and transmit dosage compliance data to external system for healthcare provider review.

The server is further configured to enable remote monitoring of patient adherence by healthcare providers by providing access to medication adherence data. The server is further configured to allow healthcare providers to intervene on detection of non-adherence of dosage compliance by generating alerts to the patient and caregiver.

The server is further configured to detect risk behaviors, including skipped doses and patterns indicating misuse, by analyzing patient medication adherence data. The server is further configured to generate and transmit alerts to healthcare providers on detecting risk behaviors, enabling timely intervention and monitoring. The server is further configured to verify if location data of the dispensing device matches against predefined location data before authorizing the release of medication. The server is further configured to ensure that the medication dispensing activities align with prescription control measures mandated by regulatory authorities.

The server is configured to use artificial intelligence to provide tailored guidance on adherence strategies and motivation. The server enables patients to immediately connect with healthcare providers in case of distress via a user device in communication with the server. The server is further configured to lock the dispensing device on detection on attempts of unauthorized access of the dispensing device. The server enables patients and healthcare providers to customize alerts via text, email, or phone calls. The server is configured to verify if the temperature and environmental conditions of the dispensing devices match predefined safe storage parameters before authorizing medication storage. The server is configured to transmit an alert to the users if the stored medication exceeds the safe storage parameters.

The dispensing device further comprises a user interface in communication with the control device. The user interface is configured to display clear instructions, alerts, and patient information in a user-friendly format. In one embodiment, the dispensing device is configured to support multiple languages for better accessibility. The system further comprises one or more user devices associated with a respective user. The user devices are in communication with the server. The server is configured to enable patients, healthcare providers and authorized caregivers to monitor adherence and set up reminders via the user device.

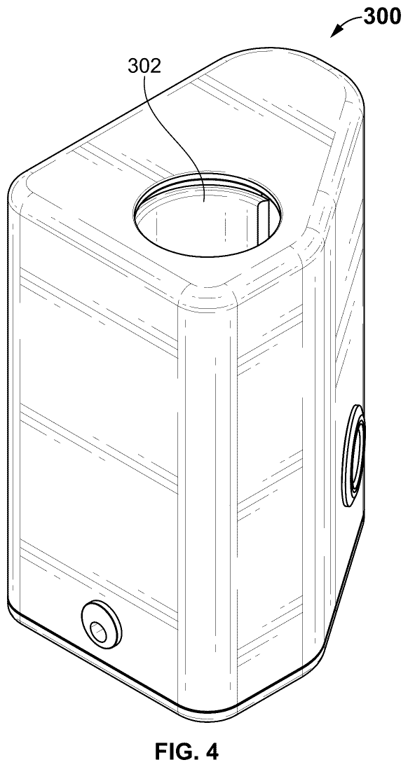

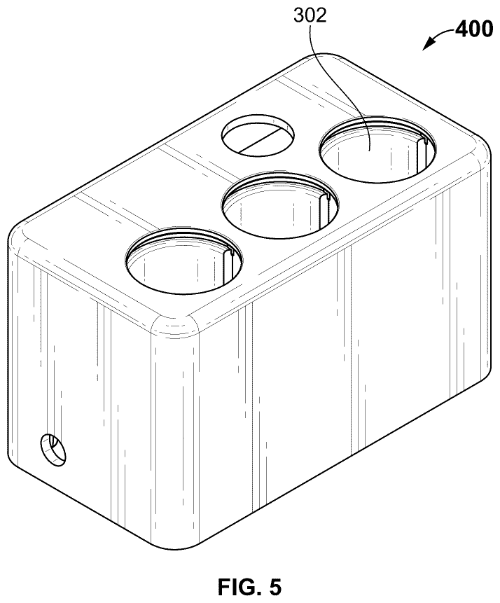

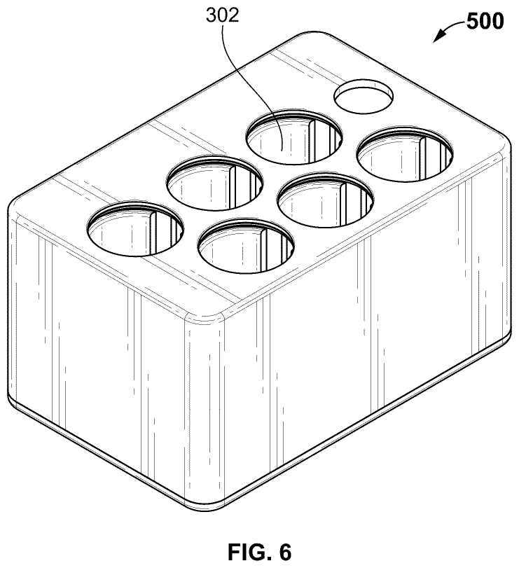

FIGS. 4, 5 and 6 illustrate different types of medication dispensing devices (300, 400, 500). The device 300 includes at least one compartment 302 for dispensing a single unit of various types of medication. The device 400 includes at least three compartments 302. The device 500 includes at least six compartments 302. The device (300, 400, 500) could include any number of compartments 302 based on the user requirement. Further, the compartments 302 of device (400, 500) could be used to dispense different types of medication or different doses.

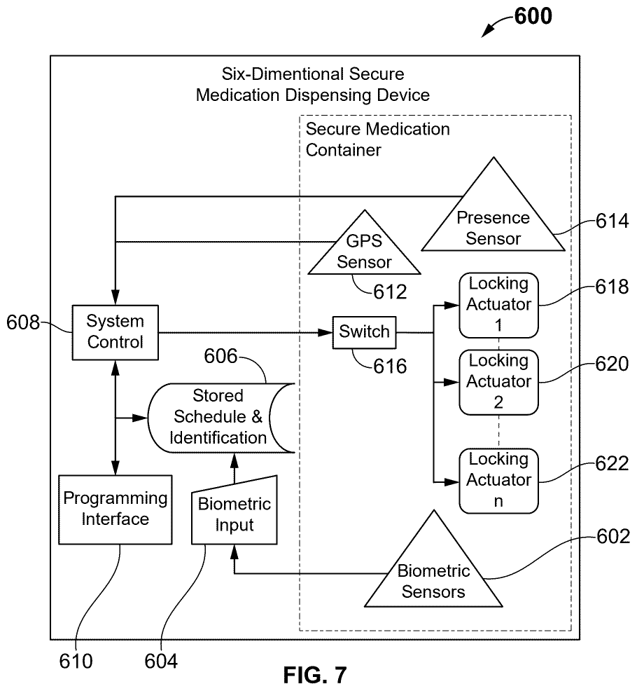

FIG. 7 is a block diagram 600 of the modular system components that comprise the physical and electronic elements within the dispensing devices (300, 400, 500, 700), according to an embodiment of the present invention. A biometric sensor 602 that is placed in the dispensing device (300, 400, 500, 700) captures the biometric input 604 such as voice recognition, fingerprint scanning, retina scanning and facial recognition. The dispensing device (300, 400, 500, 700) further receives stored schedule and identification 606. The dispensing devices (300, 400, 500, 700) further comprises a programming interface 610. The dispensing devices (300, 400, 500, 700) further comprises a system control 608 configured to control and receive data from a GPS sensor 612, a presence sensor 614, and a switch 616. The switch 616 controls the locking actuator 1 618, locking actuator 2 620, and locking actuator 622.

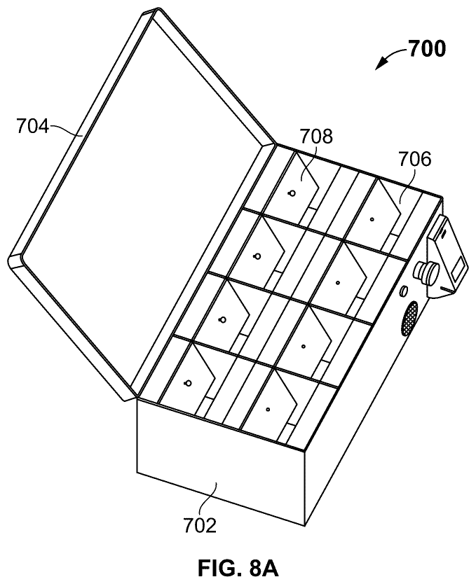

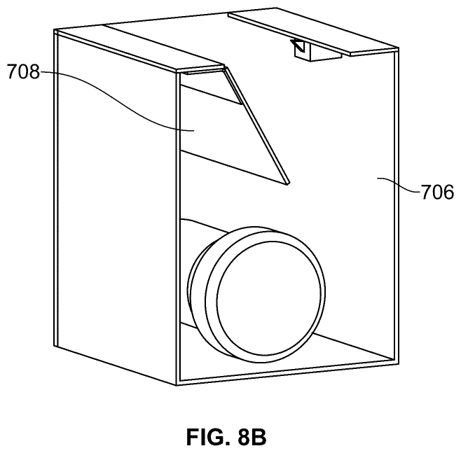

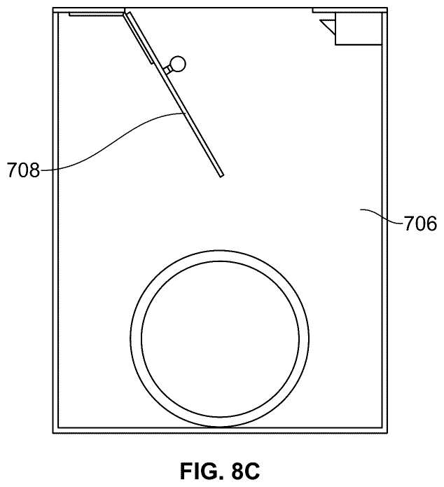

Referring to FIG. 8A, a dispensing device 700 in an open position comprises a two by four array of compartment 706. The dispensing device 700 comprises an outer housing 702, and a door 704. The outer housing 702 comprises eight individual compartments 706 with open metal lids 708. The door 704 of the outer housing 702 is held in place by a latching device.

FIG. 8B illustrates a partial exploded, perspective view of the single compartment 706 with open metal lids 708 of the dispensing device 700. FIG. 8A further shows the metal lid 708 of the single compartment 706 in open position to reveal the dispensing medical bottle of the dispensing device 700.

FIG. 8C shows a front view of the dispensing device 700, showing a storage compartment 706 with the lid 708 in open position. The dispensing device 700 comprises the outer housing 702, the door 704, a metal strip, and the latch. The outer housing 702 is designed to hold up to eight individual compartments 706 within the dispensing device 700. An array of compartments 706, each of which are capable of holding and delivering at least one dose of medication to a patient for a single day of the week. The medication dispensing device 700 further comprises a key or combination locking device. For example, the medication dispensing device 700 is at least 14 inches wide by 11 inches deep and 4 inches tall box that has a key or combination locking device. The medication dispensing device has eight access compartments 706. 708 For example, each compartment 706 opening metal lid 708 is at least 4 inches by 1.5 inches.

Further, the device 700 comprises doors 704 and latch. The various latching devices are used for locking the door 704 that secures the medication. These doors 704 are hinged in a manner to have them fall down into the compartment 706 when a latching device is actuated. When closed, the doors 704 are held in place by the latching device created by a solenoid connected to the control unit or the controller. The top of the device has metal strips that prevent the doors 704 from rotating outside of the box. This strip also provides an electronic connection point that can be used to detect tampering. In use, dispenser 700 could be used to distribute units of medications in the form of liquids, tablets, pills, sublingual film, or capsules at preprogrammed times to individuals.

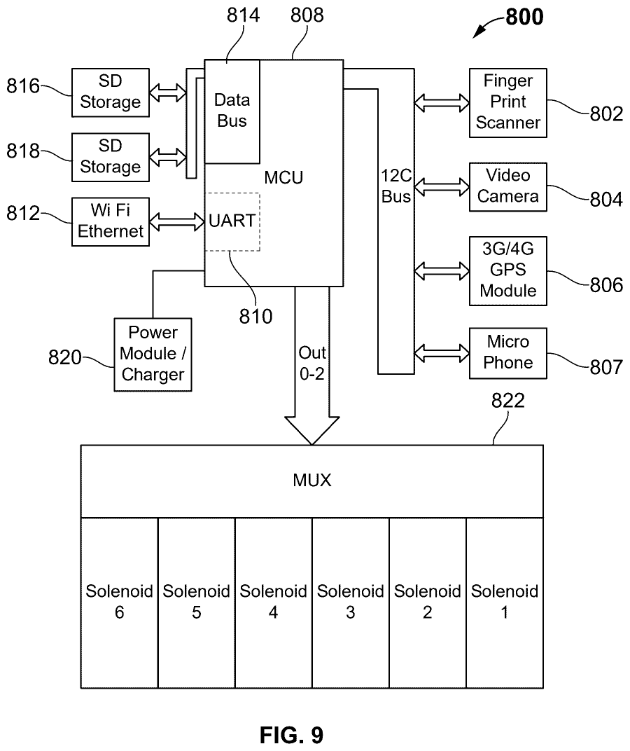

FIG. 9 shows a block diagram 800 of an electronic design component and the bus used to connect these components. The fingerprint scanner 802, camera 804 and microphone 807 are connected to the controller through a data bus that directly reads signals from input devices. These signals are captured, digitized, and stored in the memory of the controller. The figure also represents the web interface which captures the address that represents the remote location where the device will be allowed to operate. This address is thereupon converted into GPS coordinates. Upon registration of the vital inputs, the routine gets set for usage of the dispensing system subject to verification of the user's data.

The user's biometric information such as voice recognition, fingerprint scanning, retina scanning and facial recognition along with name, date of birth, and gender, and other credentials that are linked to a unique identification are captured. The biometric information is captured using the fingerprint scanner 802, camera 804, and microphone 807. The fingerprint scanner 802, camera 804, and microphone 807 are connected to the controller through a data bus that directly reads signals from input devices. These signals are captured, digitized, and stored in the memory of the controller. The microcontroller unit (MCU) 808 is a controlling device that receives the user input. The power supply for microcontroller unit (MCU) 808 is provided through power module or a charger 820. The MCU 808 uses universal asynchronous receiver/transmitter (UART) 810 for device-to-device communication. The received data from UART 810 is stored in SD storage 818, 816. The system further Wi-Fi ethernet 812 for communication and a GPS module 806 to capture the address of the remote location where the device 700 will be allowed to operate. This address is thereupon converted into GPS coordinates. Upon registration of the vital inputs, the routine gets set for usage of the dispensing system subject to verification of the user's data. Further, a multiplex (MUX) 822 is configured to forward the selected output for verification of the user's data. Identify the BUS 12C and the Out 0-2.

The medication dispensing system comprised of three tiers including a Tier 1, a Tier 2, and a Tier 3. Tier 1 is a cloud software system called the Controlled Substance Management (CSM) PolyCloud Recovery Platform (CSMPRP) that serves to manage devices located in remote clinics and offices. Tier 2 control system is a server located in these remote offices. Tier 3 Internet of Things (IoT) device dispenses medication and communicates with the client. Tier 1 controls the entire system and regulates the dispensing of medication. Tier 2 performs a control and management function for Tier 3 with instructions to open and close medication dispensing compartments. Tier 3 utilizes a specific biometric interface that enables the device to be opened in real time using a defined algorithm. The devices fall into the category of Telematics. The device has the option of integrating video and other multimedia content for communication.

Tier 1 Cloud Software is called the Controlled Substance Management (CSM) PolyCloud Recovery Platform (CSMPRP). Tier 1 controls the entire system and regulates dispensing of medication and overall access to the system. Tier 1 comprises of nine elements i.e., IoT controller management software, cloud infrastructure, applications, private social network, analytics algorithms, The Multimedia TV, database of drug sample test, dashboards, and clinic management. The IoT controller management software controls, manages, and backs up the devices on the client premise. The IoT device also backs up and stores the critical data such as fingerprints and other biometric data, which is essential to secure access and authentication. The IoT enhances the security, accessibility, and overall operational efficiency. The cloud infrastructure, which includes public (Microsoft/Amazon) elements and private server elements to provide cloud computing. The applications interact with the clients and clinicians, utilizing data captured from the system to facilitate efficient communication. The private social network facilitates therapeutic communication between clients and clinicians. The analytics algorithms that support the recovery process. The Multimedia TV is an application that manages and stores video content for broadcast over the network and directly to the patients and clients. The database of drug sample test results that can be aggregated in a de-identified manner. The sample test results that are aggregated in a de-identified form ensures easy access to critical information for monitoring and evaluating treatment effectiveness while maintaining patient privacy. The dashboards are provided for access and reporting client information, which enables clients to track the information of medication. The clinic management processes and policies information is provided to clinicians or medical staff to understand the practices employed by individuals who consumes medication. This information enables staff to provide feedback to individuals in order to shape behavior related to medication consumption compliance.

The Asendio 28 Dose Device is an advanced medication management innovation, specifically designed to optimize substance abuse treatment protocols. This device securely stores and dispenses up to 28 individual doses of medication, incorporating Artificial Intelligence (AI) to monitor patient compliance and medication usage. By utilizing AI to recognize locations of bottles or a spacial recognition routine that uses X-Y coordinates to determine if a bottle is present, data is delivered to the Tier I Engine to assess client behavior. The system captures pictures that indicate the presence of dosing bottles. These pictures are capture by the camera mounted on the top of the case. These images are captured between 5 and 15 times per second. Tier 1 software analyzes the submitted images and determines if behavior is compliant with dosing parameters. Several issues are reviewed for compliance. The first issue to review is the total bottles remaining in the device when the top is closed. This number should be consistent with the beginning number when the top is opened. The system also identifies if more than one bottle is retrieved during the dosing process. This behavior will indicate possible diversion. Another data element to review is the time that the bottles are removed. This economical and efficient device is suitable for both outpatient and remote treatment environments, proving especially useful in rehabilitation centers and methadone clinics where stringent monitoring and accurate dispensing of medication are crucial.

The Asendio 28 Dose device significantly enhances treatment outcomes by facilitating patient's adherence to dosing schedules and improving the security of medication dispensation. By integrating these features into a cost-effective design, the Asendio 28 Dose Device is a valuable tool for healthcare providers, supporting better management of substance abuse treatments and ensuring higher levels of patient and public safety.

Tier 2 Controller is a server that communicates directly with Tier 1 software and Tier 3 IoT devices. Tier 2 is critical in that it stores biometric data, enables the programming of the medication dispensing devices, and generates messages for clinicians. Tier 2 server stores rules that govern analysis and programming, stores content, and maintains the programs to control devices. This is a Wi-Fi enabled device that must be used to interface with Tier 3 device.

Tier 3 device provides safe, secure, and controlled dispensing of pharmaceuticals via a multi-dimensional secure medication dispensing procedure. Dispensing of medication via multi-factor security brought forth through a biometric that involves fingerprint, voice, retina and face recognition. The system also validates location, day, time, operator, and physical inspection.

Specifically, the device limits the medication delivery to one dose at prescribed times. The device limits access to only the prescribed patient, and that medication is accessible at a previously specified location, typically at the patient's place of residence. This invention also records distribution at the time of delivery using video technology and records instances where unauthorized individuals attempt to access the device. The onboard camera takes a photo of the available individual doses upon opening and/or closing. Each photo images and associated geocode, time and date data are uploaded to the CSMPRP for real-time enhanced artificial intelligence analysis of daily dosing compliance, and potential dosing misuse and diversion (virtual bottle check). The device provides feedback that indicates the psychosocial state of mind of the client. The dose of medication includes, but not limited to, liquid, sublingual film, patch, capsule, or tablet form or any combination.

The system includes a medication dispensing device, which uses a Microcomputer controller, such as a Raspberry Pi, that integrates electronic and mechanical components of the dispensing device. The control unit is programmed to perform registration, scheduling, verification, opening and data capture functions, which shall be elaborated further.

The system including the IoT device operates the dispensing device to provide a dose of medication to the patient at the appropriate predetermined time. For example, the controller can be programmed with a medication dispensing schedule which incorporates a data store comprising the predetermined time and day of the week to operate the dispenser. The data store of the medication dispensing program includes patient data, biometric information, and the Global Positioning System (GPS) coordinates of the approved dispensing location. The system also retrieves and transmits data regarding use of the system or biometric data collected from the client.

The initial function of the CSM controller is client registration, which captures the biometric data and associates it with a specific identification number. The web interface signals the device to record the fingerprint signature, picture, and a voice print. These biometric identifiers are then converted into digital representations and stored in the controller's memory. The controller also has the optional capability to back up this data in the CSMPRP located in the cloud.

The data storage of the medication dispensing program or controller can also comprise caregiver data. The caregiver can be anyone such as a family member or nurse or jail administrator who is charged with taking care of the patient. In such cases where a caregiver is necessary to administer the medication to the patient, the caregiver can be allowed to engage the system to receive the medication to be administered to the patient. Caregiver data can comprise the identification number, photo, the finger print data and the voice recognition data, retina data or the like. Through such information, the caregiver can gain access to the medication to be administered to the patient.

To accomplish the above functions of identification of the correct individual for dispensing medicine, at the correct day of the week and the time of the day, the outer housing 702 of the medical dispensing device is provided with a touch pad which is a fingerprint scanner 802 and is used to capture digital identification from the user of the system and is a critical component that initiates the registration process.

The fingerprint scanner 802 can be in communication with the controller to verify the identity of the patient. For instance, fingerprint scanner 802 can comprise a touch pad that can provide a place for the user to place a finger. Fingerprint scanner 802 can then read the fingerprint and compare it with the stored data to confirm that the individual trying to receive the doses of medication is in fact the intended recipient.

Upon capturing a fingerprint signature, a photo and/or video through the camera 804 that resides next to the fingerprint scanner 802. Besides the camera 804 and the scanner 802, a speaker and microphone are provided for enabling the system to capture a voice print which is digitized and stored in the system.

The fingerprint scanner 802, camera 804 and microphone 807 are connected to the controller through a data bus that directly reads signals from input devices. These signals are captured, digitized, and stored in the memory of the controller.

An integrated Global Positioning System (GPS) 806 locator provided also on the outer housing 702 of the medication dispensing device enables the captured address of the device, converted into GPS coordinates, and which represents the remote location where the device will be allowed to operate via the data input through the web interface and transmitted to the controller. Optionally, a GSM connection with the cellular network will enable GPS 806 and interactive communication. The IoT devices also communicate with the CSM Controller located at the facility.

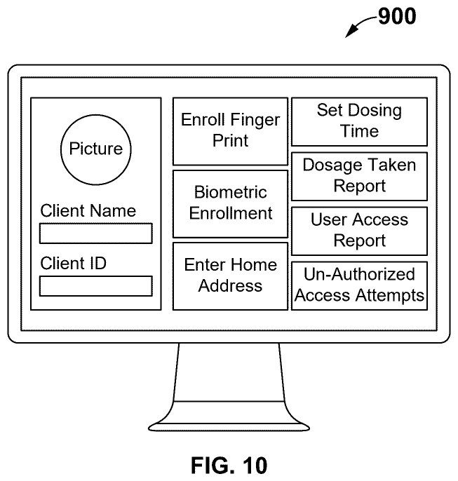

FIG. 10 depicts a user interface 900 of the dispensing device (300, 400, 500, 700) The screen is a web-based application that connects to the uniquely identified dispensing device via Wi-Fi or USB. There are six areas depicted on the screen: ID recording to read the biometric input from the patient in the form of fingerprint, retina, or voice; day of week access to indicate the days that the device should be opened; time of day access; location specific access to identify the exact location that the Dispensing Device (300, 400, 500, 700) can be accessed; specific numbers of failed attempts to access the device (300, 400, 500, 700); and, the number of unique individuals attempting to access the device (300, 400, 500, 700). The interface also includes a mechanism to view authorized access by individuals.

The operation of the device (300, 400, 500, 700) requires several routines:

### Configuration Routine

The CSM Controller must be configured for each premise facility, such as a clinic or doctor's office. This routine requires the specific storage requirements, behavioral routines, interfaces, IoT device options, IP addresses and passwords to be programmed for each controller.

### Registration Routine

The fingerprint system is the critical component that initiates the registration process and is used to capture digital identification from the user of the system. Upon capturing a fingerprint signature, a photo and/or video is captured through the camera 804 that resides next to the fingerprint scanner 802. Finally, the user is asked to speak a phrase into the speaker to enable the system to capture a voice print which is digitized and stored in the system. These digital representations of biometric information are stored on the CSM Controller and on the IoT device. This information is backed up in the central cloud system.

### Verification Routine

The user of the system initiates the entry process by placing a finger or thumb on the fingerprint scanner 802. The scanner sends an alert to the system that requests a digital signal from the reader. Upon capturing a digital fingerprint, this digital representation is compared to the digital signature stored in the controller's memory. If the digital comparisons match a positive signal, the corresponding signal is sent indicating that there is an identity match. Should the digital comparison not indicate a match, an alert is sent to the user indicting that there has been a mismatch. Upon this first phase of verification, additional points of verification can be achieved by using facial verification and/or location matching.

During the course of facial verification, for each attempt, a picture is taken that is saved in the memory and registered as either a valid or invalid access attempt. The alert sent by the fingerprint scanner 802 also triggers the GPS module 806 to access the current location of the device. Face verification and/or location matching can be used as multifactor location elements. A similar process will occur for voice verification.

Upon satisfaction of the required verification elements, the appropriate device compartment (302, 706) is opened whereupon usability of the medication application is taken up through the scheduling process.

### Scheduling Routine

The scheduling routine involves syncing the program of the device (300, 400, 500, 700) with the function of the system that translate to inputting the specific days and times of operation into the system through programming of the device (300, 400, 500, 700). The number of days programmed into the system equates to the number of medication doses in the container. For each day, a specific time range is provided as input and stored in the device's memory.

The location captured during the registration process is stored as the location of use at which the device (300, 400, 500, 700) must be made stationary to enable the opening operation. Scheduling information is collected using the web interface and communicated to the dispensing device using the Wi-Fi or USB interface to ensure safe start of operations coinciding with the opening stage.

### Opening Routine

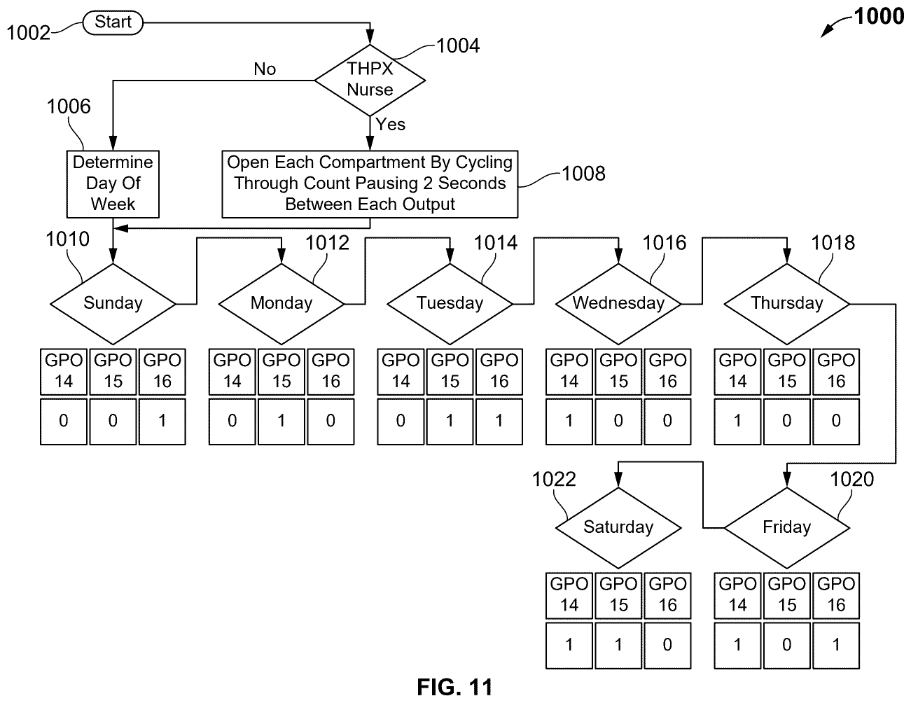

FIG. 11 depicts a logic flowchart 1000 representing the process of verification of the day of the week once the location, fingerprint and/or facial verification and/or voice recognition is completed, for opening the particular dispensing compartment (302, 706).

### Consumption Analysis Routine

Analysis of uninhibited access to medication-The onboard camera takes a photo of the available individual doses upon opening and/or closing. Each photo image and associated geocode, time and date data are uploaded to the CSMPRP for real-time enhanced artificial intelligence analysis of daily dosing compliance, and potential dosing misuse and diversion (virtual bottle check).

The process starts at step 1002. In step 1004, the routine involves a multiplexer being used to interact with the control device and select the appropriate solenoid for opening of a specific compartment (302, 706). The opening routine utilizes several factors for verification.

At step 1006, after location, fingerprint and/or facial verification, a routine to verify the day of the week is employed.

At step 1008, after the opening state is verified, the device controller uses a three-input multiplexor to select one of seven opening outputs (step 1010, 1012, 1014, 1016, 1018, 1020, and 1022). The controller opens each compartment by cycling through count pausing 2 seconds between each output.

Each opening output is connected to a solenoid or similar device. Once the solenoid is activated, the latch is contracted, which allows the door 704 to release to expose the medication container. Only the solenoid in the compartment (302, 706) for the day selected is activated by the routine. This compartment (302, 706) remains open until the user selects the container found in the compartment (302, 706). The container is returned to the compartment (302, 706) after use and the door 704 to the compartment is closed. As noted, upon opening the specific compartment (302, 706), a picture can be optionally taken for security and archival purposes.

After medication is dispensed and consumed, the empty medication container is returned to the unique compartment (302, 706), and the compartment (302, 706) is closed so that the entire dispensing cycle can be repeated. Data on the state of the opening transaction is collected and stored. The system or sever verifies if location data of the dispensing device matches against predefined location data before authorizing the release of medication. The system or server ensures that the medication dispensing activities align with prescription control measures mandated by regulatory authorities.

The system enables interactive, real-time data to be shared using the GSM network or other network provided. The IoT device also can be fitted with a Narcan device that is used in case of opioid overdose. A battery backup is optionally provided either inside the device or as an external component.

Apart from the above verification, for individuals attempting to access the dispensing device, a physical review of the device using a six-point inspection plan is performed on each visit where take-home doses are granted. This is considered as the sixth verification point in the multi-factor security inspection.

It is to be noted that the data is captured on every attempted transaction, whether successful or not. In each such transaction, the location, facial print, finger print, voice print and location of the device are collected. This information is saved in the device's memory and is retrieved from the device by the web application when connected to the host computer.

The host web applications interrogate the device for all attempted access information, including finger prints, facial prints, voice recognition, location information, day and time of dispensing and the time that the compartment (302, 706) lid 708 is opened. The web app also uploads and organizes face prints to enable operator verification to ensure the intended client is permitted access to the device.

As a fail-safe, the system also records attempted access to the device in order to facilitate overall system security. This is a human inspection point that provides a visual observation to verify accurate and reliable operation. The device also tracks and records the amount of time that the metal lid 708 of the compartment (302, 706) was opened. One concern regarding dispensing is that the medication should be consumed within a short period of time to prevent sharing of medication or split dosing.

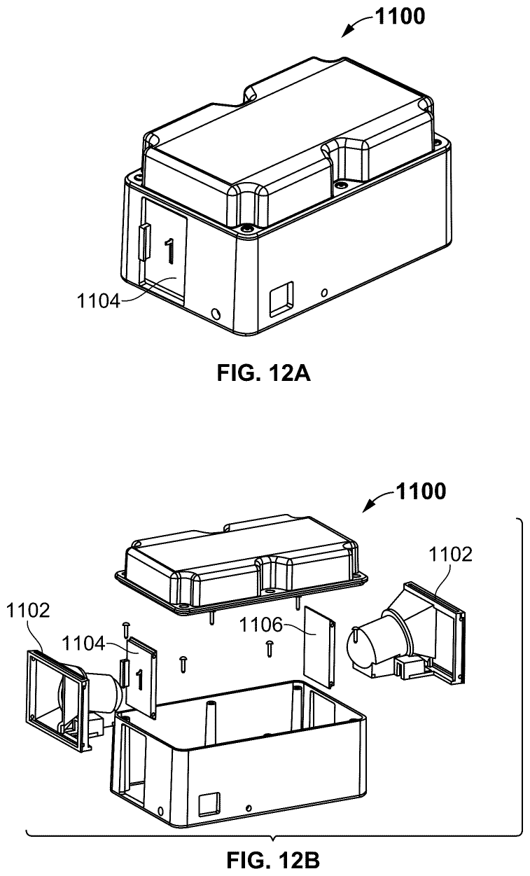

Referring to FIG. 12A to 12C, in another embodiment, a two-dose modified Polycase container assembly 1100 is disclosed. The two-dose modified Polycase container assembly 1100 comprises a dose holder 1102, a first door 1104, a second door 1106, one or more solenoids, and at least one finger print reader. The two-dose modified Polycase container assembly 1100 comprises two dose unit. Each dose unit is placed in an individual dose holder 1102. Referring to FIG. 12 C, the assembly 1100 have dose holder 1102 at a first door 1104 and at a second door 1106.

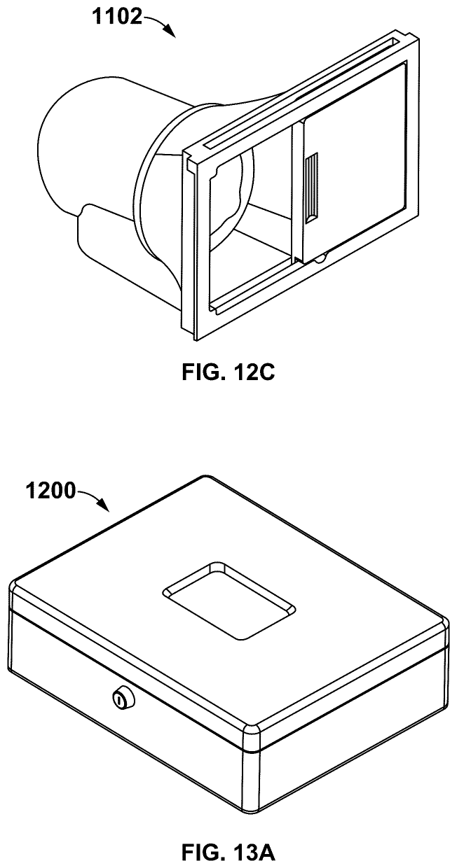

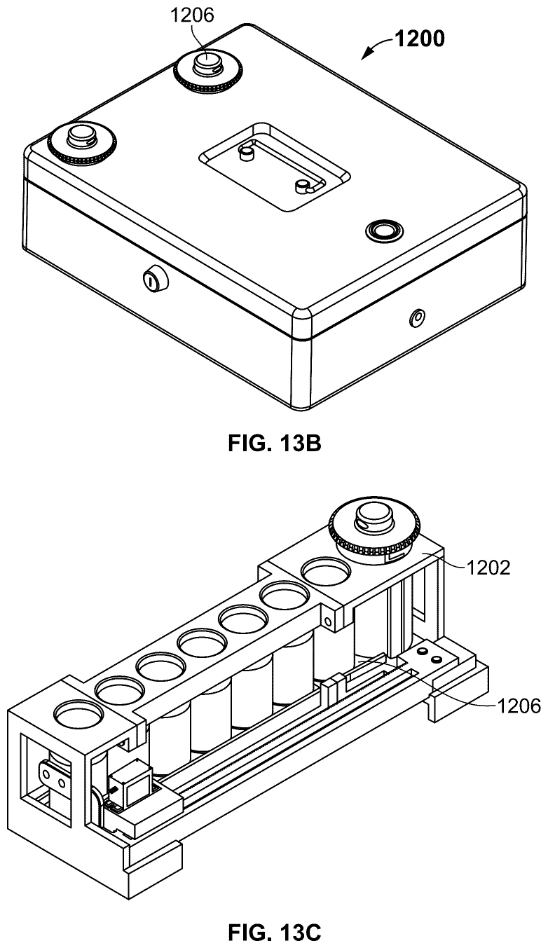

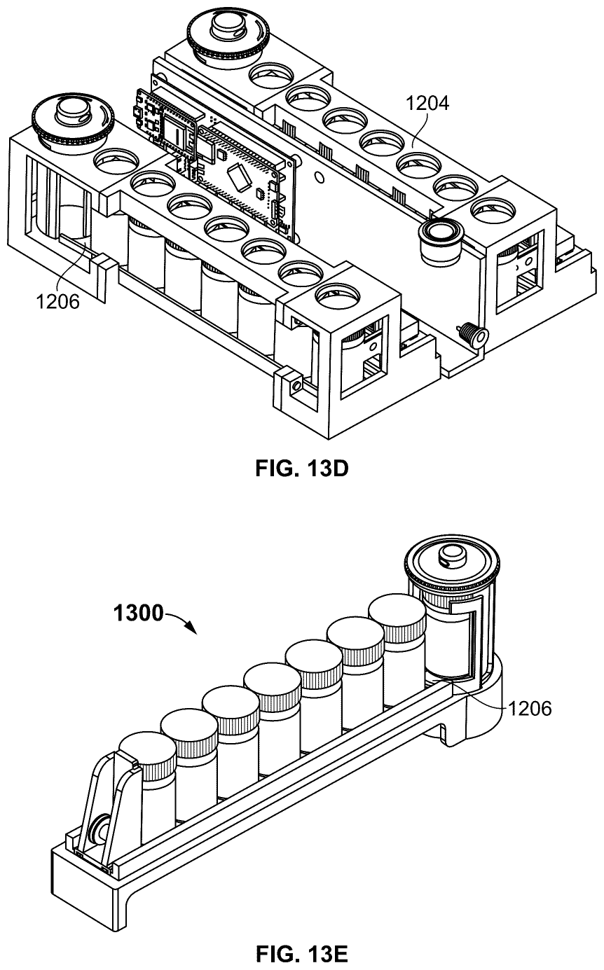

Referring to FIG. 13A to 13E, in another embodiment, a metal box 1200 with seven dose units 1202 and fourteen dose units 1204 is disclosed. Each dose unit dispense the doses via a pull up chamber discharge design 1206. The pull up chamber discharge design 1206 is powered via a servo motor. Further, seven dose units 1202 and fourteen dose units 1204 utilizes the servo motors with printed circuit board, finger print reader and power connection port. The servo motor is configured to control the dispensing of doses while the fingerprint reader serves as a security measure to ensure that only authorized individuals can access the box. Referring to FIG. 13C, the seven dose units 1202 are arranged in the metal box 1200. Referring to FIG. 13D, the fourteen dose units 1204 are arranged in the metal box 1200. Referring to FIG. 13E, in other embodiment, the seven dose units pusher device 1300 using a pull up chamber discharge design 1206 and 3D printed base. Further, the dose units could be scaled up from seven dose units to twenty-one or twenty-eight or the required number of doses required by the users.

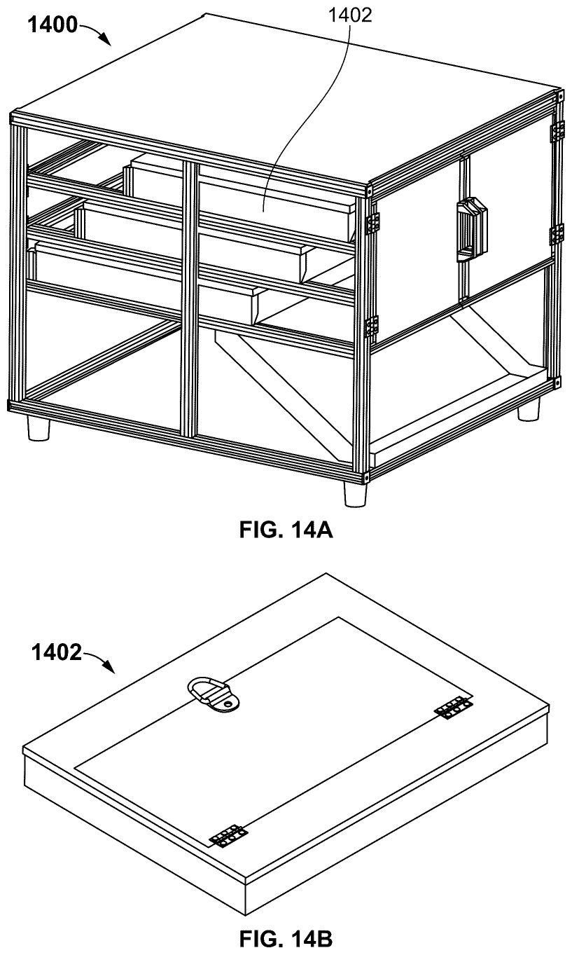

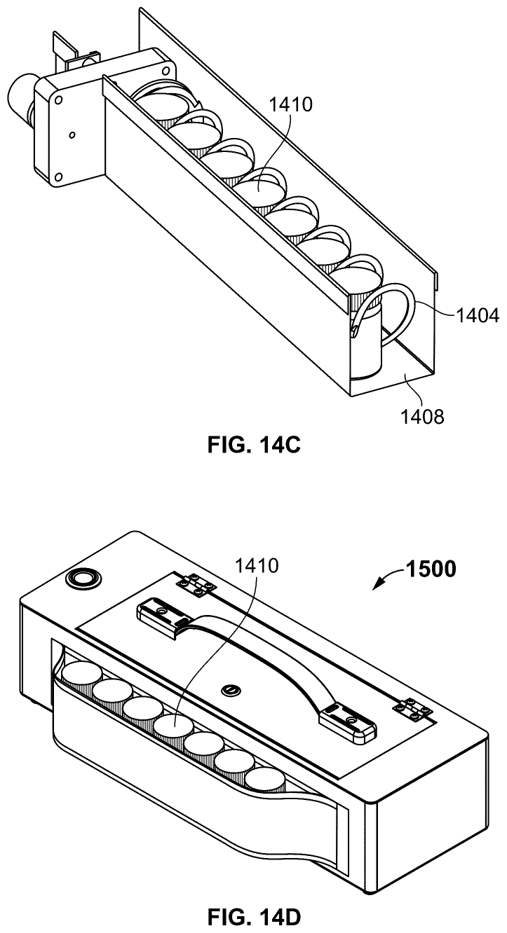

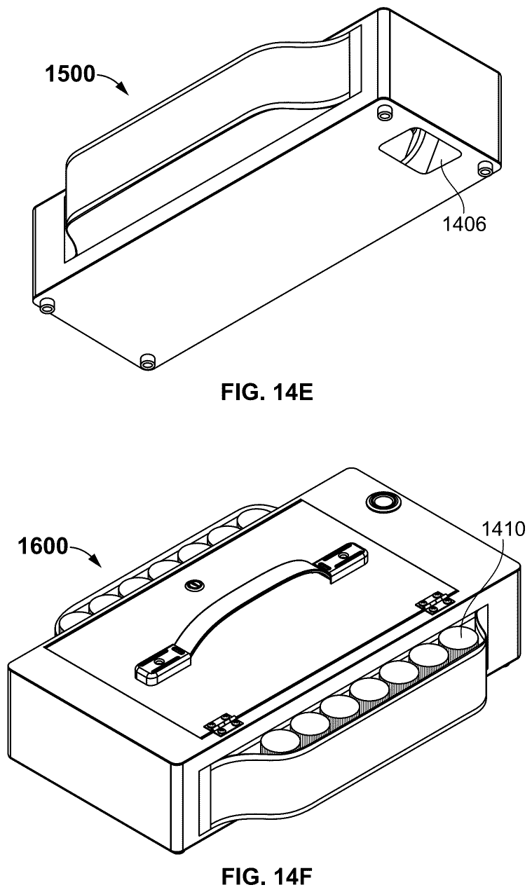

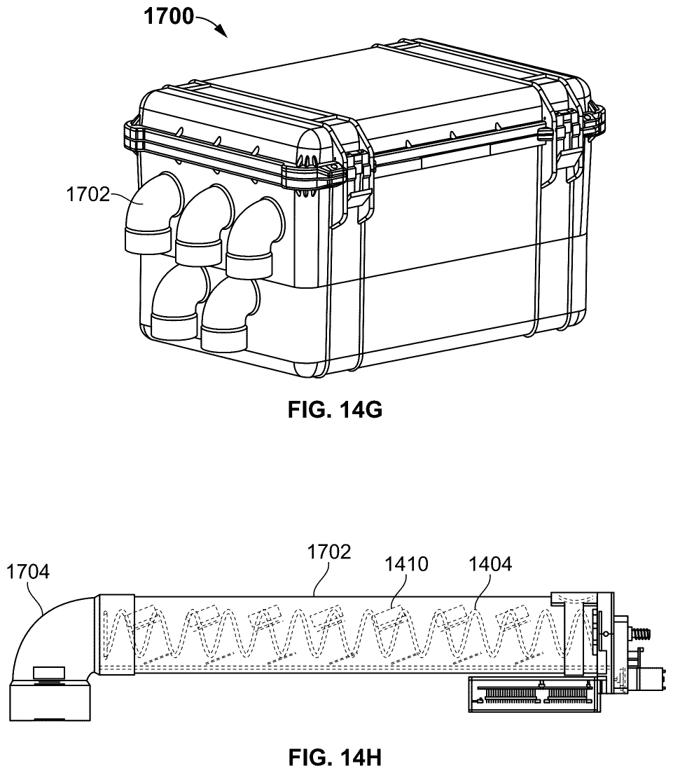

Referring to FIG. 14A to 14F, in another embodiment, a remote monitoring device 1400. The remote monitoring device 1400 comprises a tray 1402, a vending machine and motor 1404, and a bottom discharge portion 1406. The tray 1402 comprises six channels 1408. Each channel 1408 comprises seven dose unit 1410. Further, each tray 1402 has forty-two dose unit 1410. The vending machine coil and motor 1404 is disposed in each channel 1408 of the tray 1402. The vending machine coil and motor 1404 facilitates to store and distribute dose unit 1410. In one embodiment, the remote monitoring device 1400 comprises three trays 1402 that can store one hundred and twenty-six individual dose unit 1410. The individual dose unit 1410 are dispensed and retrieved through the bottom discharge portion 1406 of the trays 1402 and directed via a ramp to the front of the remote monitoring device 1400. The dose unit 1410 is then removed by the administrator and given to the patient for consumption. The remote monitoring device 1400 used for remote dispensing at locations where there is no Medication Assisted Treatment (MAT) facility. The locations, includes, but not limited to, jail, emergency care center or inpatient facility. Further, the trays 1402 are removed and returned to the home clinic for refill. The dose units 1410 can be refill on a weekly basis.

Referring to FIG. 14C, the single channel 1408 of the tray 1402 comprises seven dose unit 1410 with the vending machine coil and motor 1404. Further, the dose unit 1410 is bottle with medication. Referring to FIG. 14D, the tray 1402 comprises seven dose unit 1410 is dispensed using vending machine and motor 1404. The remote monitoring device 1400 is made up of light grade aluminium, and 3D plastic components. Further, the device 1400 comprises finger print reader and special biometric locking mechanisms. Referring to FIG. 14D, in one embodiment, the device 1400 comprises of seven dose unit tray 1500. Referring to FIG. 14E, the rear part of the seven-dose unit tray 1500 comprises bottom discharge portion 1406 that enables to distribute the dose unit 1410.

Referring to FIG. 14F, in other embodiment, the device 1400 comprises of fourteen dose unit tray 1600 configured with two vending machine coil and motor 1404. The medication dispensing method can be either at the bottom discharge portion 1406 of fourteen dose unit tray 1600 the enclosure using coil and motor 1404 or via a pull up chamber discharge design 1408.

Referring to FIG. 14G, in another embodiment, an alternative design case 1700 used for transportation comprises five PVC pipes 1702 with vending machine coils and motor 1404. The case can be provided with 65 doses. Referring to FIG. 14H, the alternative design case 1700 using the vending machine coil and motor 1404 enclosed inside a PVC pipe 1702 and elbow 1704. The dose units 1410 are assembled inside the PVC pipe 1702. Further, the vending machine coil and motor 1404 enable to dispense the dose unit 1410 via elbow 1704.

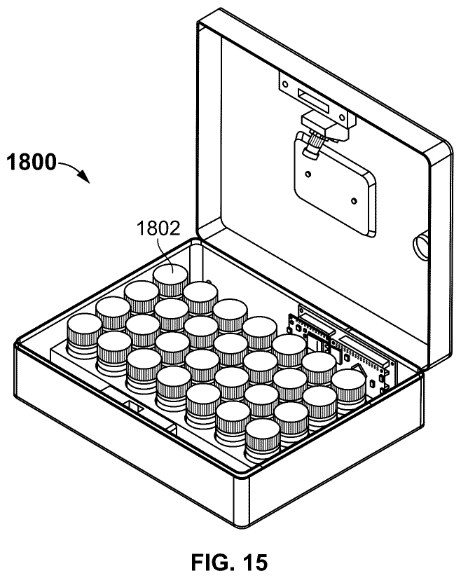

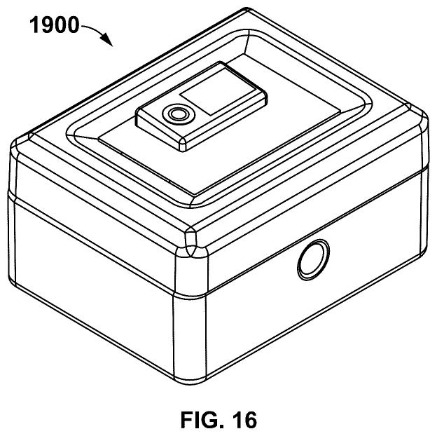

Referring to FIG. 15, in another embodiment, twenty-eight dose units housed in a metal enclosure device 1800. The device 1800 is used for individual patients. The device 1800 further comprises a finger print reader, an electronic locking mechanism, a battery/backup power supply, and a camera monitoring sensors to monitor the presence of the dose. Further, the camera integrated into the enclosure enables automatic taking of snapshots of the current available medications. Each image is geocoded, including time and date stamped data. The user can have access to all twenty-eight dose units 1802; but the users are allowed to remove and consume only one dose unit 1802 on a daily basis. The dose units 1802 are monitored through utilizing a programmed sensor system that monitors dosing activities to record the user's behavior. The device further comprises 3D printed parts, sensors, biometric locks, and electronics.

Referring to FIG. 16, a standard metal cash box 1900 with video screen and microphone included for telemedicine.

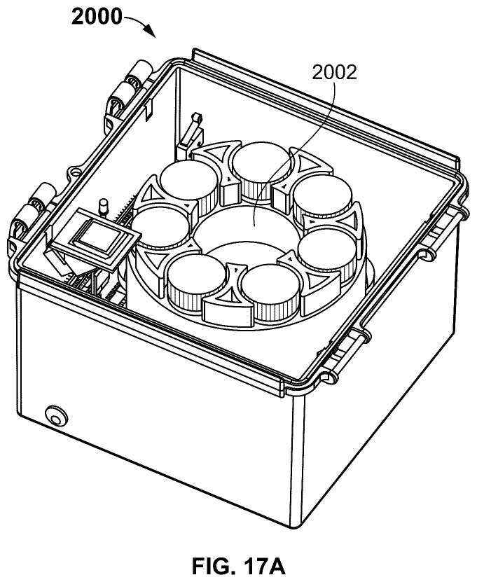

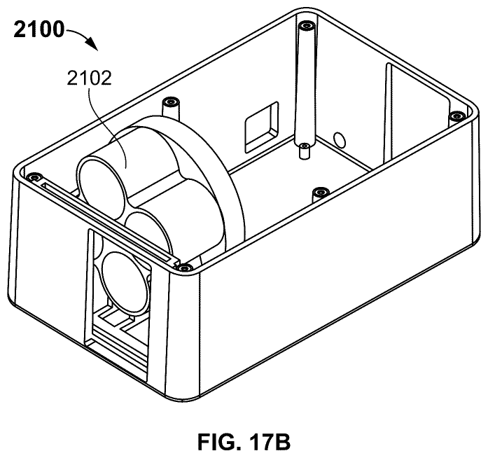

Referring to FIGS. 17a and 17b are carousel designs placed in Polycase containers 2000. Referring to FIG. 17A, the Polycase container 2000 comprises a six dose in a single carousel 2002. Referring to FIG. 17B, the Polycase container 2100 comprises dual carousel 2002 design to dispense six doses.

The foregoing descriptions of specific embodiments of the present invention have been presented for purposes of illustration and description only. They are not intended to be exhaustive or to limit the present invention to the precise forms disclosed, and obviously modifications remain possible, in particular from the point of view of the design of the various elements or by substitution of equivalent methods, without thus departing from the scope of protection of the invention.

What is claimed is:

1. A system for managing dispensing of medication, comprising:
one or more dispensing devices configured to store and dispense different types and doses of medication, wherein the dispensing device is configured to receive biometric data, identification data of a user including a patient and a caregiver, image data, video data, location data of dispensing devices, and dosage data of patient;
one or more Internet of things (IoT) devices in communication with the dispensing device configured to:
store biometric data and identification data of a caregiver and patient, and location data, and
enable the dispensing device to provide a dose of medication to the patient at a predefined time on receiving authentication of biometric data, identification data of at least one of patient and caregiver data and location data of dispensing device;
at least one database configured to store patient data including biometric data, identification data, medical data and dosage data, location data comprising information related to location approved for operation of the dispensing device, and caregiver data including biometric data and identification data, wherein the biometric data and identification data includes voice data, image data and video data, wherein the image data includes images of compartments to record available doses of medication at the compartments upon opening and closing the dispensing device, wherein the video data includes videos of environment of the dispensing on unauthorized access of the dispensing device, and
at least one server in communication with the dispensing devices, IoT devices and the database, wherein the server comprises one or more artificial intelligence modules, and a IoT control module, wherein the server is configured to:
receive patient data and caregiver data from the dispensing devices and link the patient data and caregiver data to respective unique identities, and receive dosage data of patients,
receive image data and video data of the user on accessing the dispensing device,
compare the identification data and biometric data of the user with stored identification data and biometric data for sending authentication to IoT devices and dispensing devices for dispensing medication at predefined time, and
analyze image data and dosage data, and determine adherence of patient to dosage compliance.

2. The system of claim 1, wherein the dispensing device comprises:
a housing comprising one or more compartments, wherein each compartment comprises at least one lid;
a door to provide access within the housing, wherein the door is configured to move between an open position and a closed position, and
a latching device configured to lock and unlock the door, and a metal strip comprises an electronic connection point to detect tampering of the dispensing device, wherein, in an open position of the door, the metal strip is configured to hold the door in place and, on actuating the latching device to close the door, the door is released to close the housing using a solenoid connected to the latching device, wherein the dispensing device is configured to dispense medication in the form of liquids, tablets, pills, sublingual films, or capsules.

3. The system of claim 2, wherein the dispensing device further comprises:
one or more sensors comprises a biometric sensor, wherein the biometric sensor is configured to capture the biometric data by at least one of voice recognition, fingerprint scanning, retina scanning and facial recognition;
at least one camera device disposed at the housing of the dispensing device configured to capture image data including images of compartments to record available doses of medication at the compartments upon opening and closing the door;
at least one video device disposed at the housing of the dispensing device configured to capture video data including videos of environment of the dispenser device on unauthorized access of the dispensing device;
at least one GPS module configured to capture location data;
a switch in communication with the latching device;
at least one memory configured to store the biometric data, image data, video data, and location data of the user;
a power source to operate the dispensing device, and
a control device in communication with the sensors, camera device, video device, GPS module, switch, power source and memory, wherein the control device is configured to:
receive and store biometric data, image data and video data of the user, and location data of the dispensing device, and
transmit the biometric data, image data and video data of the user, and location data of the dispensing device to the server for registration of the user and for authentication on accessing the dispensing device.

4. The system of claim 3, wherein the biometric sensor comprises a touch pad including a finger print scanner configured to capture fingerprint data.

5. The system of claim 1, wherein the determining of dosage compliance involves:
determining if the compartments are same as that of an initial number of compartments, and amount of medication before accessing the device and after closure of the dispensing device.

6. The system of claim 1, wherein server is configured to:
manages video data for broadcasting to respective user including patient, healthcare provider or caretaker, and
provide a dashboard comprising patient data and dosage data to enable patients and caretakers to track the information of medication.

7. The system of claim 1, wherein the dispensing device comprises a multi- factor authentication system.

8. The system of claim 1, wherein the server is configured to:
track whether the patient takes medication as prescribed by analyzing patient data, dosage data, and image data from the dispensing device;
generate and transmit an alert to healthcare providers on non-adherence of dosage compliance, and
automatically record dispensing events and transmit dosage compliance data to an external system for healthcare provider review.

9. The system of claim 1, wherein the server is configured to:
enable remote monitoring of patient adherence by healthcare providers by providing access to medication adherence data, and
allow healthcare providers to intervene on detection of non-adherence of dosage compliance by generating alerts to the patient and caregiver.

10. The system of claim 1, wherein the server is configured to:
detect risk behaviors, including skipped doses and patterns indicating misuse, by analyzing patient medication adherence data;
generate and transmit alerts to healthcare providers on detecting risk behaviors, enabling timely intervention and monitoring;
Verify if the temperature and environmental conditions of the dispensing devices match predefined safe storage parameters before authorizing medication storage, wherein the server is configured to transmit an alert to the users if the stored medication exceeds the safe storage parameters;
verify if location data of the dispensing device matches against predefined location data before authorizing release of medication, and
ensure that the medication dispensing activities align with prescription control measures mandated by regulatory authorities.

11. The system of claim 1, wherein the dispensing device is configured to provide verbal reminders and step-by-step instructions to assist patients with medication adherence.

12. The system of claim 1, wherein the dispensing device further comprises:
a user interface in communication with the control device configured to display clear instructions, alerts, and patient information in a user-friendly format, wherein the dispensing device is configured to support multiple languages for better accessibility.

13. The system of claim 1, further comprises one or more user devices associated with the respective user, wherein the user device is in communication with the server, wherein the server is configured to enable patients, healthcare providers and authorized caregivers to monitor adherence and set up reminders via a user device in communication with the server.

14. The system of claim 1, wherein the server is configured to:
use artificial intelligence to provide tailored guidance on adherence strategies and motivation;
enables patients to immediately connect with healthcare providers in case of distress via the user device;
locks the dispensing device on detection on attempts of unauthorized access of the dispensing device, and
allows patients and healthcare providers to customize alerts via text, email, or phone calls.
